SEQUENCE LISTING (10 sequences)

SEQ ID NO: 1 - DNA, 20 bp, bacteria
taagtccsgcaacgagcgca

SEQ ID NO: 2 - DNA, 20 bp, bacteria
gtgacgggcggtgwgtacaa

SEQ ID NO: 3 - DNA, 19 bp, fungi
caactttcgatggtaggat

SEQ ID NO: 4 - DNA, 20 bp, fungi
atcgtcttcgatcccctaac

SEQ ID NO: 5 - DNA, 21 bp, Staphylococcus sp.
tttagggctagcctcaagtga

SEQ ID NO: 6 - DNA, 20 bp, Staphylococcus sp.
cacttctaagcgctccacat

SEQ ID NO: 7 - DNA, 24 bp, Staphylococcus aureus
gcatcagtgacagagagtgttgac

SEQ ID NO: 8 - DNA, 23 bp, Staphylococcus aureus
ttatactcgtggtgctggtaagc

SEQ ID NO: 9 - DNA, 20 bp, bacteria
gcaatcgctaaagaactaag

SEQ ID NO: 10 - DNA, 20 bp, bacteria
gggaccaacataacctaata

(19) United States
(12) Patent Application Publication
(10) Pub. No.: US 2010/0255474 A1
(43) Pub. Date: Oct. 7, 2010

(54) Method for Detecting Bacteria and Fungi

(76) Inventors: Stefan Russwurm, Jena (DE); Julien Landre, Munchen (DE); Marc Lehmann, Jena (DE)

Correspondence Address: Lewis Kohn & Fitzwilliam LLP, 10935 Vista Sorrento Parkway, Suite 370, San Diego, CA, 92130, US

(21) Appl. No.: 12/676,550

(22) PCT Filed: Sep. 3, 2008
(86) PCT No.: PCT/EP08/07197

§ 371 Date: Apr. 21, 2010

(30) Foreign Application Priority Data
Sep. 4, 2007 (DE) 10 2007 041 864.9

(51) Int. Cl. C12Q 1/68

(52) U.S. Cl. 435/6

(57) ABSTRACT

The present invention relates to methods and means for determining pathogenic fungi in a sample material, e.g. blood. In the method, the bacterial DNA is initially enriched from the total DNA of the sample material, and then the enriched DNA is amplified with specific primer pairs. Detection of the obtained amplicons allows the accurate identification of bacteria and fungi contained in the sample material and of their resistances. The methods and means of the invention allow an early diagnosis of inflammatory diseases, in particular involving non-detected infection (SIRS), and of infectious diseases such as sepsis, spontaneous bacterial peritonitis and endocarditis.

### CROSS REFERENCES

This application is a United States National Stage Application claiming priority under 35 U.S.C. 371 from International Patent Application No. PCT/EP2008/007197 filed Sep. 3, 2008, which claims the benefit of priority from German Patent Application Serial No. 102007041864.9 filed Sep. 4, 2007, the entire contents of which are herein incorporated by reference.

### DESCRIPTION

The invention relates to the determination of bacteria, fungi and the antibiotic or antimycotic resistances thereof in sample material by the detection of specific nucleic acid sequences.

The determination of pathogenic micro-organisms such as bacteria and fungi in a sample material is highly important in numerous areas and particularly in medicine. Bacterial contaminations in thrombocyte concentrates are, for instance, a crucial factor for transfusion-associated morbidity and mortality and are currently the most frequent infectious complication in transfusion medicine. An early detection of microbial pathogens associated with infections is indispensable for a rapid and effective antimicrobial therapy, for example in patients with sepsis, spontaneous bacterial peritonitis (SBP), and endocarditis. Moreover, in this context, the increasing number of infections with unknown pathogens in hospitals, particularly in intensive care units, which often are caused by a compromised immune defense of the patients and by the increasingly frequent invasive treatments associated with a higher risk of infection, but also may be a consequence of poor hygiene, present a serious problem.

The micro-organisms causing these infections are unknown in most cases and may belong to numerous different genera and species. In order to be able to rapidly identify any existing contaminations or infections, it is therefore necessary to simultaneously test the sample material for as many candidate micro-organisms as possible. This is highly important particularly in cases of clinial samples, as effective therapeutic treatment, such as an antibiotic therapy adapted to the respective pathogen, depends on the analytic result.

The detection of the microbial pathogens of an infectious disease is frequently performed using a blood culture or some other culture of a body fluid, followed by biochemical typing and detection of antibiotic resistances. However, up to the present a high percentage of blood cultures are being tested false-negative so that patients are subjected to an antibiotic treatment without established microbiologic evidence. (Bosshard et al., 2003, CID 37:167-172; Gauduchon et al., 2003, J. Clin. Microbiol. 41:763-766; Grijalva et al., 2003, Heart 89:263-268).

Apart from microbiologic diagnostics, there are additional specific detection techniques for some special applications such as, e.g., protein biochemical antigen detection using direct immunofluorescence techniques, agglutination tests or ELISA for the diagnosis of sepsis or meningitis caused by meningococci, Haemophilus influenzae, Group NB streptococci (McLellan et al., 200Infect Immun. 69(5):2943-2949) or pneumococci.

One rapid and elegant method for diagnosing bacterial infections is the polymerase chain reaction (PCR) where specific regions of the bacterial genome (e.g., highly variable 16S or 23S rDNA regions (Anthony et al. 2000), tRNA genes in the 16S-235 rDNA spacer region as well as other pathogen-specific genes such as, e.g., adhesins, hemolysins or various toxins (Bélanger et al., 2002, J. Olin. Microbiol. 40(4):1436-1440; Depardieu et al., 2004, J. Clin. Microbial. 40(4):1436-1440; Kaltenboeck and Wang, 2005, Adv. Clin. Chem. 40:219-259; Patel et al., 2007, J. Clin. Microbiol. 35(3):703-707; Sakai et al., 2004, J. Clin. Microbiol. 42(12):5739-5744)) are amplified. Sequencing (of, for example, 16S rDNA PCR amplicons) may follow for further differentiation (Unemo et al., 2004, J. Olin. Microbiol. 42(7):2926-2934). WO 97/07238 discloses a method for detecting fungi such as Candida and Aspergillus using primers for the amplification of all types of fungal ribosomal 18S rDNA. None of these detection and differentiation methods in molecular biology is currently used routinely or was established as a standard method.

In order to achieve the sensitivities required for clinical samples, the template DNA necessary for this purpose is obtained from bacteria which were isolated from positive blood cultures. In case of non-culturability of the pathogens to be detected (for example if taking the blood sample is preceded by antibiotic therapy), the molecular-biologic detection accordingly also remains negative. In the absence of a preculturing step, the low ratio of prokaryotic to human DNA in clinical samples may be increased by enrichment of the bacterial nucleic acids. Corresponding methods are described, for example, in EP-A-1 400 589, WO-A-2005/085440, and WO-A-2006/133758.

Techniques suitable for species differentiation meanwhile also include Raman/FTIR techniques (Fourier Transform Infrared Spectroskopy; Rebuffo et al., 2006, Appl. Environ. Microbiol. 72(2):994-1000; Rebuffo-Scheer et al., 2007, Circulation 111; 1352-1354) and SERS techniques (Surface Enhanced Raman Scattering; Kahraman et al., 2007, Appl. Spectrosc. 61(5):479-485; Naja et al., 2007, Analyst 132(7):679-686), which over the past decade have achieved a level of sensitivity allowing to obtain even spectra of individual living cells. In practice, however, up to 1,000 cells in pure culture are necessary in order to obtain spectroscopic data for differentiations. This renders these techniques unsuited for rapid diagnosis of specific sepsis pathogens (Kirschner, 2004, Doctoral Thesis Univ. Berlin).

Diagnosis of pathogens is followed by an antibiotic therapy adapted to the pathogen. If determining the causative pathogen or of the existing resistances, respectively, is not possible, however, it is necessary to undertake an empirical and time-consuming therapy using broad range antibiotics.

The prior art, however, shows several drawbacks. Thus, e.g., blood cultures remain negative in cases of sepsis involving non-culturable pathogens or when blood is taken following pre-treatment with (broad range) antibiotics (up to 90% of all bacterial sepsis cases are blood culture-negative). Accordingly, a subsequent molecular-biologic differentiation based on the extraction of prokaryotic DNA from positive blood cultures is theoretically only possible in ≦10% of sepsis cases. Moreover, due to the vast spectrum of pathogens and due to the occurrence of pathogen types that have previously not been described, the generation of individual, highly specific primers and probes is only of limited use for the unambiguous identification of a pathogen. Differentiation on a type level and furnishing of an antibiogram requires a plurality of selected primers/probes and a combination of PCR and hybridization techniques. As not all antibiotic resistances are genome-coded or have a known coding, genotypical detection of resistance markers is of limited success and has to be supplemented with a time-consuming phenotypical test (Gradelski et al., 2001, J. Clin. Microbiol. 39(8):2961-2963). As described above, this in turn requires that the pathogen is culturable. In case of multiple infections, moreover, sequencing of 16S-rDNA amplicons may lead to non-interpretable results due to sequence superpositions.

The object of the present invention, therefore, is to provide methods and means which allow a simple and reliable determination of bacteria and fungi that may be present in a sample material.

It is another object of the present invention to provide methods and means allowing early determination of pathogenic bacteria and fungi in a sample material so as to allow rapid initiation of a therapeutic treatment adapted to the detected pathogens.

According to the invention, this object was achieved by a method for determining bacteria and fungi contained in a sample material, wherein said method comprises the following steps:
a) enriching bacterial and fungal DNA contained in the sample material;
b) amplifying the DNA obtained in step a) using primer pairs selected from at least two of groups (i) to (vii):
   (i) at least one primer pair which is suited for the specific amplification of a region of a particular nucleic acid sequence that is specific for a plurality of bacteria families;
   (ii) at least one primer pair which is suited for the specific amplification of a region of a particular nucleic acid sequence that is specific for a plurality of fungus families;
   (iii) at least one primer pair which is suited for the specific amplification of a region of a particular nucleic acid sequence that is specific for a selected bacteria genus;
   (iv) at least one primer pair which is suited for the specific amplification of a region of a particular nucleic acid sequence that is specific for a selected bacteria species;
   (v) at least one primer pair which is suited for the specific amplification of a region of a particular nucleic acid sequence that is specific for the expression of a selected antibiotic or antimycotic resistance;
   (vi) at least one primer pair which is suited for the specific amplification of a region of a particular nucleic acid sequence that is specific for a selected fungus genus; and
   (vii) at least one primer pair for the specific amplification of a region of a particular nucleic acid sequence that is specific for a selected fungus species; and, optionally,
c) detecting the amplicons formed in step b);
   and the kit for the determining bacteria and fungi contained in a sample material, wherein the kit comprises:
a) means for enriching bacterial and fungal DNA contained in the sample material;
b) means for amplifying DNA, wherein the means include primer pairs selected from at least two of groups (i) to (vii):
   (i) at least one primer pair which is suited for the specific amplification of a region of a particular nucleic acid sequence that is specific for a plurality of bacteria families;
   (ii) at least one primer pair which is suited for the specific amplification of a region of a particular nucleic acid sequence that is specific for a plurality of fungus families;
   (iii) at least one primer pair which is suited for the specific amplification of a region of a particular nucleic acid sequence that is specific for a selected bacteria genus;
   (iv) at least one primer pair which is suited for the specific amplification of a region of a particular nucleic acid sequence that is specific for a selected bacteria species;
   (v) at least one primer pair which is suited for the specific amplification of a region of a particular nucleic acid sequence that is specific for the expression of a selected antibiotic or antimycotic resistance;
   (vi) at least one primer pair which is suited for the specific amplification of a region of a particular nucleic acid sequence that is specific for a selected fungus genus; and
   (vii) at least one primer pair for the specific amplification of a region of a particular nucleic acid sequence that is specific for a selected fungus species, and, optionally,
c) means for detecting the amplicons obtainable with the primer pairs of b).

In accordance with the invention, it was surprisingly found that the combination of a step of enriching bacterial and fungal DNA from total DNA and of an amplification step involving selected primer pairs allows not only to considerably enhance detection sensitivity for individual bacteria and fungi, but that in this way it is also possible to detect numerous different genera and species of bacteria and fungi in parallel.

The object of the present invention, therefore, is a method for determining bacteria and fungi contained in a sample material, said method comprising the following steps:
a) enriching bacterial and fungal DNA contained in the sample material;
b) amplifying the DNA obtained in step a) using primer pairs selected from at least two of groups (i) to (vii):
   (i) at least one primer pair which is suited for the specific amplification of a region of a particular nucleic acid sequence that is specific for a plurality of bacteria families;
   (ii) at least one primer pair which is suited for the specific amplification of a region of a particular nucleic acid sequence that is specific for a plurality of fungus families;
   (iii) at least one primer pair which is suited for the specific amplification of a region of a particular nucleic acid sequence that is specific for a selected bacteria genus;
   (iv) at least one primer pair which is suited for the specific amplification of a region of a particular nucleic acid sequence that is specific for a selected bacteria species;
   (v) at least one primer pair which is suited for the specific amplification of a region of a particular nucleic acid sequence that is specific for the expression of a selected antibiotic or antimycotic resistance;
   (vi) at least one primer pair which is suited for the specific amplification of a region of a particular nucleic acid sequence that is specific for a selected fungus genus; and
   (vii) at least one primer pair which is suited for the specific amplification of a region of a particular nucleic acid sequence that is specific for a selected fungus species; and, optionally,
c) detecting the amplicons formed in step b).

Another object of the invention is a diagnostic kit for determining bacteria and fungi contained in a sample material, wherein the kit comprises:
a) means for enriching bacterial and fungal DNA contained in the sample material;
b) means for amplifying DNA, wherein the means include primer pairs selected from at least two of groups (i) to (vii):
   (i) at least one primer pair which is suited for the specific amplification of a region of a particular nucleic acid sequence that is specific for a plurality of bacteria families;
   (ii) at least one primer pair which is suited for the specific amplification of a region of a particular nucleic acid sequence that is specific for a plurality of fungus families;
   (iii) at least one primer pair which is suited for the specific amplification of a region of a particular nucleic acid sequence that is specific for a selected bacteria genus;
   (iv) at least one primer pair which is suited for the specific amplification of a region of a particular nucleic acid sequence that is specific for a selected bacteria species;
   (v) at least one primer pair which is suited for the specific amplification of a region of a particular nucleic acid sequence that is specific for the expression of a selected antibiotic or antimycotic resistance;
   (vi) at least one primer pair which is suited for the specific amplification of a region of a particular nucleic acid sequence that is specific for a selected fungus genus; and
   (vii) at least one primer pair for the specific amplification of a region of a particular nucleic acid sequence that is specific for a selected fungus species, and, optionally,
c) means for detecting the amplicons formed using the primer pairs of b).

The sample material includes any material in which bacteria and fungi may occur. Usually the sample material is an environmental sample, a food sample or a biological sample, for example a clinical sample. The biological sample may be a plant sample, the biological or clinical sample, however, typically is a human or animal sample, in particular a sample from a mammal. Typically, the sample is a human or animal tissue sample or body fluid. The tissue sample may be a biopsy, for instance. Preferably the sample is a body fluid or a product derived therefrom, for example full blood, serum, plasma, thrombocyte concentrate, cerebro-spinal fluid, liquor, urine and pleural, ascites, pericardial, peritoneal or synovial fluid. The sample material may be obtained in a usual manner; for example, a clinical sample may be obtained by biopsy, taking blood or puncture.

The enrichment of prokaryotic and fungal DNA from the sample material is carried out following extraction of total DNA from the sample material. Accordingly, the kit of the invention may also contain means for the extraction of total DNA from the cells contained in the sample material, as is described below by way of example. For the extraction of the total DNA prior to the actual enrichment step, the cells present in the sample, including the bacterial and fungal cells contained in it, are initially disrupted or lyzed. Disruption and lysis of cells may take place in a manner known per se, for example mechanically using high-pressure homogenizers or preferably using glass beads and vortex treatment, chemically by use of solvents, detergents or alkali, enzymatically by use of lytic enzymes, or combinations of these techniques. Enzymatic lysis of bacterial cells is preferred, with lysozyme or mutanolysin typically being used for digestion, advantageously in combination with alkali, detergents and proteolytic enzymes. The digestion of fungal cells is typically performed mechanically, for example by vortexing with glass micro-beads, advantageously in combination with alkali, detergents and further proteolytic enzymes. However, digestion may also be performed enzymatically with, e.g., zymolase being used as the enzyme. Extraction of total DNA may then take place in a manner known per se by adsorption to a DNA-binding matrix. Kits for isolating total DNA are commercially available and may be used in accordance with the manufacturers' specifications. For example, components required for isolating total DNA are contained in the LOOXSTER® kit for enrichment of bacterial and fungal DNA from total DNA. Following elution of the matrix, a sample with total DNA is obtained in which the DNA is present in aqueous solution.

The actual enrichment of bacterial and fungal DNA from the sample with total DNA takes place using means that specifically bind the bacterial and fungal DNA, in particular using proteins and polypeptides that specifically bind the bacterial and fungal DNA. Typically, enrichment of prokaryotic and fungal DNA is carried out according to the methods described in EP-A-1 400 589, WO-A-2005/085440 and WO-A-2006/133758 which are incorporated herein by reference. The methods and means described therein allow to increase the low ratio of prokaryotic and fungal DNA relative to other DNA contained in the sample, in particular human or animal DNA. Here, the DNA, which is present in solution after the preparation of total DNA, is contacted with a protein or a polypeptide capable of binding to non-methylated CpG motifs. As non-methylated CpG motifs occur markedly more frequently in bacterial and fungal DNA than in higher eukaryotic DNA such as human or animal DNA, bacterial and fungal DNA are preferably bound to these proteins or polypeptides. The protein or polypeptide may be coupled to a support such as microparticles. In this way, the formed protein/polypeptide DNA complex may easily be separated from human or animal DNA, for instance by filtration, centrifuging or magnetic methods. The selective binding of prokaryotic and fungal DNA to these proteins and polypeptides results in an enrichment of the DNA by a factor of 5 or more. Kits for the enrichment of bacterial and fungal DNA from total DNA, which also include means for the preparation of total DNA, are commercially available under the trade name LOOXSTER® (SIRS-LAB GmbH, 07745 Jena, Germany).

The DNA enriched in bacterial and fungal DNA is subsequently amplified by non-quantitative or quantitative amplification methods, in particular non-quantitative or quantitative PCR (Polymerase Chain Reaction) in the presence of a set or pool of different primer pairs which allow for a specific amplification of regions of particular nucleic acid sequences that are specific for bacteria, fungi, or antibiotic or antimycotic resistances. Nucleic acid sequences which are specific for bacteria, fungi or selected genera and species thereof or for antibiotic and antimycotic resistances may be obtained from publicly accessible gene libraries such as Gen Bank and TIGR, or other commercial gene libraries, and a person skilled in the art will be capable of designing corresponding appropriate primers routinely and without undue burden, for example using the publicly accessible website “Primer3” (see, e.g. http://frodo.wi.mit.edu/cgi-bin/primer3/primer3_www.cgi of the MIT) or other commercial software.

In accordance with the invention, amplification is carried out using primer pairs from at least two of the above groups (I) to (vii), which are selected such that upon amplification they result in amplicons having a predetermined, previously known length. The presence of amplicons of the expected length formed using at least one primer pair (i) then indicates the presence of bacteria; the presence of amplicons formed using at least one primer pair (ii) indicates the presence of fungi; the presence of amplicons formed using at least one primer pair (iii) indicates the presence of at least one particular bacteria genus; the presence of amplicons formed using at least one primer pair (iv) indicates the presence of at least one particular bacteria species; the presence of amplicons formed using at least one primer pair (v) indicates the presence of at least one particular antibiotic or antimycotic resistance; the presence of amplicons formed using at least one primer pair (vi) indicates the presence of at least one particular fungus genus; and the presence of amplicons formed using at least one primer pair (vii) indicates the presence of at least one particular fungus species.

Primer pairs of group (I) are generic primers which specifically hybridize to a highly preserved nucleic acid sequence which is common to a plurality of or all bacteria families. Bacteria families which occur particularly frequently in infections and contaminations and the presence of which may therefore advantageously be tested with primer pairs of group (i) are, for example, Pseudomonadaceae, Enterobacteriaceae, Streptococcaceae, Staphylococcaceae, Listeriaceae, Neisseriaceae, Pasteurellaceae, Legionellaceae, Burkholderiaceae, Bacillaceae, Clostridiaceae, Moraxellaceae, Enterococcaceae and/or Bacteroidaceae. Examples of nucleic acid sequences that are highly preserved in all bacteria families are the sequences of the 16S rDNA gene, of the 23S rRNA gene and of the 16S/23S interspace region. One example of a primer pair which specifically hybridizes to the nucleic acid sequence of the gene for the bacterial ribosomal 16S rDNA and may be used in accordance with the invention is the primer pair:

| | |
|---|---|
| 5′-TAAGTCCSGCAACGAGCGCA-3′ | (SEQ ID NO: 1) |
| (forward primer) | |
| 5′-GTGACGGGCGGTGWGTACAA-3′ | (SEQ ID NO: 2) |
| (reverse primer) | |

wherein S represents the bases C or G, and W represents the bases A or T. The detection of amplicons that are formed after amplification with at least one primer pair (i) generally indicates the presence of bacteria in the sample material.

Primer pairs of group (ii) are generic primers which hybridize to a highly preserved nucleic acid sequence that is common to a plurality of or all fungus families. Families of fungi which occur particularly frequently in contaminations and infections and whose presence may therefore advantageously be tested with primer pairs of group (ii) are, for example, fungi of the family Trichocomaceae and of the Candida family. One example of a nucleic acid sequence highly preserved in all fungus families is the sequence of the gene for the fungal 18S rDNA. One example of a primer pair which specifically hybridizes to the nucleic acid sequence of the gene for the fungal 18S rDNA and which may be used in accordance with the invention, is the primer pair:

| | |
|---|---|
| 5′-CAACTTTCGATGGTAGGAT-3′ | (SEQ ID NO: 3) |
| (forward primer) | |
| 5′-ATCGTCTTCGATCCCCTAAC-3′ | (SEQ ID NO: 4) |
| (reverse primer) | |

which results in amplicons having a length of about 670-690 by upon amplification. The detection of amplicons which are formed following amplification with at least one primer pair (ii) generally indicates the presence of fungi in the sample material.

Primer pairs of group (iii) are primers which hybridize to a highly preserved nucleic acid sequence that is common to a plurality of or all bacteria species of a particular genus but not to all bacteria genera of a family. Bacteria genera which occur particularly frequently in infections and contaminations and the presence of which may therefore advantageously be tested with primer pairs of group (iii) are, for example, bacteria of the genera Staphylococcus spp, Streptococcus spp, Enterococcus spp, Escherichia spp, Pseudomonas spp, and Enterobacter spp. One example of a primer pair which hybridizes genus-specifically to DNA of bacteria of the genus Staphylococcus and may be used in accordance with the invention, is the primer pair:

| | |
|---|---|
| 5′-TTTAGGGCTAGCCTCAAGTGA-3′ | (SEQ ID NO: 5) |
| (forward primer) | |
| 5′-CACTTCTAAGCGCTCCACAT-3′ | (SEQ ID NO: 6) |
| (reverse primer) | |

which specifically hybridizes to a nucleic acid sequence of the 23S region that is specific for Staphylococci and results in a staphylococcus-specific amplicon having a length of 418 by upon amplification. The detection of amplicons that are formed after amplification with at least one primer pair (iii) indicates the presence of a particular genus of bacteria in the sample material. For example, amplicons with the above primer pair indicate the presence of bacteria of the genus Staphylococcus.

Primer pairs of group (iv) are primers which hybridize to a preserved nucleic acid sequence that is common to a plurality or all bacteria of a particular species but not to all bacteria species of a genus. Bacteria species which occur particularly frequently in contaminations and infections and the presence of which may therefore advantageously be tested with primer pairs of group (iv) are, for example, bacteria species of the above-mentioned bacteria genera, e.g. Staphylococcus aureus, Staphylococcus haemolyticus, Streptococcus pneumoniae, streptococci of the Viridans group, Enterococcus faecium, Enterococcus faecalis, Morganella morganii, Klebsiella pneumoniae, Klebsiella oxytoca, Escherichia coli, Burkholderia cepacia, Prevotella melaminogenica, Stenotrophomonas maltophilia, Pseudomonas aeruginosa, Proteus mirabilis, Enterobacter aerogenes and Enterobacter cloacae. Non-limiting examples of preserved species-specific nucleic acid sequences are the emp gene of Staphylococcus aureus, the irp2 gene of Escherichia coli, and the ureA gene of Klebsiella pneumoniae. One example of a primer pair which hybridizes species-specifically to DNA of bacteria of the species Staphylococcus aureus and may be used in accordance with the invention, is the primer pair:

| | |
|---|---|
| 5′-GCATCAGTGACAGAGAGTGTTGAC-3′ | (SEQ ID NO: 7) |
| (forward primer) | |
| 5′-TTATACTCGTGGTGCTGGTAAGC-3′ | (SEQ ID NO: 8) |
| (reverse primer) | |

which specifically hybridizes to the nucleic acid sequence of the emp gene and results in the formation of an amplicon having a length of 948 bp. The detection of amplicons that are formed after amplification with at least one primer pair (iv) indicates the presence of a particular bacteria species in the sample material. For example, amplicons with the above primer pair indicate the presence of bacteria of the species Staphylococcus aureus.

Primer pairs of group (v) are primers which allow the amplification of a nucleic acid sequence that is specific for a selected antibiotic or antimycotic resistance, e.g. the nucleic acid sequence of a corresponding resistance gene. Antibiotic and antimycotic resistances which occur particularly frequently in contaminations and infections and the presence of which may therefore advantageously be tested with primer pairs of group (v) are, for example, methicillin resistances, e.g. methicillin-resistant Staphylococcus aureus (MRSA). Examples of highly preserved nucleic acid sequences that are specific for the expression of antibiotic and antimycotic resistances are nucleic acid sequences of the genes for the methicillin resistance, such as mecA. One example of a primer pair which specifically hybridizes a gene participating in the methicillin resistance, mecA, and which may be used in accordance with the invention, is the primer pair:

| | |
|---|---|
| 5′-GCAATCGCTAAAGAACTAAG-3′ | (SEQ ID NO: 9) |
| (forward primer) | |
| 5′-GGGACCAACATAACCTAATA-3′ | (SEQ ID NO: 10) |
| (reverse primer) | |

which specifically hybridizes to a nucleic acid sequence of the mecA gene and results in the formation of an amplicon having a length of 222 bp. The detection of amplicons which are formed after amplification with at least one primer pair (v) indicates the presence of antibiotic or antimycotic resistances for the bacteria or fungi contained in the sample material. For example, the use of the above primer pairs indicates a methicillin resistance.

In analogy with the primer pairs described in the foregoing for bacteria genera or bacteria species, primer pairs of group (vi) are primers which hybridize to a highly preserved nucleic acid sequence that is common to a plurality or all fungus species of a genus but not to all fungus genera of a family. Genera of fungi which occur particularly frequently in contaminations and infections and the presence of which may therefore advantageously be tested with such primers are, for example, fungi of the genera Aspergillus and Candida. Correspondingly, primer pairs of group (vii) are primers which hybridize to a highly preserved nucleic acid sequence that is common to a plurality or all fungi of a particular species but not to all fungus species of a genus. Fungus species which occur particularly frequently in contaminations and infections and the presence of which may therefore advantageously be tested with such primers are, for example, the species Aspergillus fumigatus and Candida albicans. The detection of amplicons formed after amplification with the at least one primer pair (vi) or (vii) thus indicates the presence of a particular genus or species of fungi in the sample material.

The families, genera and species of bacteria and fungi as well as the antibiotic and antimycotic resistances that may be tested with the above combinations are basically not limited, and the families, genera and species named above as well as the mentioned primer pairs merely represent exemplary embodiments for the method of the invention. As was explained in the foregoing, nucleic acid sequences which are specific for bacteria, fungi or particular genera and species thereof or for antibiotic and antimycotic resistances may be obtained from publicly accessible gene libraries, and corresponding appropriate primers may be constructed routinely and without undue burden by a person skilled in the art.

The amplification step of the method of the invention is performed in parallel with at least two primer pairs, i.e., as a multiplex method. In accordance with the invention it is possible to use a random combination of groups (i) to (vii) of primer pairs for amplification. In accordance with a preferred embodiment, at least primer pairs from groups (i) and (ii) are used for amplification. In accordance with other preferred embodiment, at least primer pairs from groups (i) and (ii), (iii), (iv) and (v); (i), (iii) and (iv); (i), (ii), (iii) and (iv); and (i), (ii), (iii), and (v) are used for amplification. In a particularly preferred manner, amplification is performed with primer pairs from groups (i) to (vi), in a quite particularly preferred manner with primer pairs from all groups (i) to (vii). The number of the primer pairs altogether and of primer pairs employed from each group is not subject to any particular restrictions and essentially depends only on the micro-organisms presumed to be present in the sample to be examined and on the therapy relevance and the desired scope, in particular the desired accuracy of detail of the examination results. Thus it is not required, e.g. in testing clinical samples for infections to test for all of the Streptococcus species as the therapy pattern for all Streptococci is essentially the same. The method of the invention may readily be performed with 150 different primer pairs or more and is usually performed with at least 10, preferably with at least 20, and in a particularly preferred manner with at least 30 and more different primer pairs.

Multiplex amplification may take place by means of random, non-quantitative or quantitative amplification methods. In a preferred manner, amplification is performed by means of non-quantitative PCR or (quantitative) real-time-PCR (in the following also referred to as qPCR). The present invention shall in the following be described for PCR without, however, being limited thereto.

Multiplex PCR kann be performed in one or several reaction vessels. For practical reasons, multiplex PCR is frequently performed in several reaction vessels, particularly if a large number of different primer pairs are used in the amplification. In general, every reaction vessel then contains different primer pairs. Multiplex PCR is preferably performed in one or two reaction vessels.

In accordance with a preferred embodiment, amplification is carried out by non-quantitative PCR. Amplification is carried out in a manner known per se to the skilled person under suitable amplification conditions, i.e., cyclically changing reaction conditions which allow for in vitro reproduction of the starting material having the form of nucleic acids. In general, the PCR consists of a number of 25 to 50 cycles that are performed inside a thermocycler. Following initialization, each cycle consists of the steps of denaturation, primer hybridization (annealing), and elongation (extension) that are performed at temperatures which depend on the selected primer pairs and the employed enzymes. In the reaction mixture, the building blocks for the selectively reproduced nucleic acid sections, the amplicons, are present in the form of the deoxynucleotide triphosphates together with the primer pairs that attach to complementary regions in the starting material, and a suitable, usually heat-resistant polymerase. Suitable amplification conditions, e.g. cation concentrations, pH value, volume, duration and temperature of the single, cyclically repeated reaction steps in dependence on the selected primer pairs and the employed enzymes, are routinely selected by the person skilled in the art.

In one advantageous embodiment of the present invention, amplification is performed under conditions under which the amplicons are labelled with a detectable marker. This may be achieved, e.g., by the nucleotides employed in the PCR including one or several nucleotides provided with a detectable marker, which are incorporated into the amplicon during amplification and allow the detection of this amplicon by way of this marker. In accordance with one embodiment, radioactive markers, e.g. ³²P, ¹⁴C, ¹²⁵I, ³³P or ³H, are used as the detectable marker. In accordance with a preferred embodiment of the invention, non-radioaktive markers, in particular color or fluorescence markers, enzyme or immune markers, quantum dots or other molecules detectable, e.g., as a result of a bindung reaction such as biotin, are used as detectable markers, the detection of which may take place in a manner known per se to a person skilled in the art. Particularly preferred are color and fluorescence markers as well as biotin markers. Still more preferred, biotinylated nucleotides are used in the PCR, e.g. biotin-dUTP, which results in a biotinylated amplicon that may be detected, e.g., by its binding to streptavidin. The selection of suitable markers is routine work for a person skilled in the art.

In accordance with another embodiment, amplification is performed by means of real-time PCR (qPCR). The method of qPCR is known per se to a person skilled in the art and is described in detail, e.g., in US-A-2006/0099596, In qPCR, the formation of the PCR products in every cycle of the PCR is monitored. To this end, the amplification is usually measured in thermocyclers equipped with suitable means for monitoring fluorescence signals during the amplification. Devices suitable for this purpose are commercially available, e.g. under the trade name Roche Diagnostics LightCycler™.

The detection of the formed amplicons may take place both with non-labelled and labelled amplicons.

If the obtained amplicons do not contain any detectable markers, their detection may take place, e.g., based on their known size and by separation by gel electrophoresis and subsequent visualization, e.g. by staining with ethidium bromide and using UV light. Gel electrophoresis may take place in a manner known per se, e.g. by agarose gel electrophoresis or polyacrylamide gel electrophoresis (PAGE). In a preferred manner, gel electrophoresis is performed on agarose gels. In gel electrophoresis, the separated amplicons are compared with a ladder of DNA markers for size determination. The comparison suitably takes place with a DNA ladder including a mixture of the DNA fragments expected in the amplification. The presence of amplicons in the amplification mixture whose size conforms with the DNA markers indicates the presence of the micro-organisms for which these fragment lengths are specific.

In an advantageous embodiment, the amplicons generated in the PCR contain a detectable marker. In this case, detection is preferably performed with hybridization techniques (arrays), e.g. with a microarray such as a DNA microarray. Preferably, detection takes place using a microarray. In this case the amplification mixture obtained in the PCR, which in the presence of bacteria and fungi in the tested sample contains labelled, e.g. biotinylated amplicons, is contacted with a set of polynucleotide-based probes which contain nucleic acid sequences that are complementary to the amplicons obtained, in a given case, in the PCR and that have been applied to a solid support, e.g. a glass support, in defined positions of a raster (“spots”), under conditions allowing hybridization. The selection of parameters for adjusting of suitable hybridization conditions is generally known to the skilled person. These are physical and chemical parameters which may influence the establishment of a thermodynamic equilibrium of free and bound molecules. The skilled person is capable of adjusting the time period of the contact between probe and sample molecules, cation concentration in the hybridization buffer, temperature, volume, as well as concentrations and ratios of the hybridized molecules in the interest of optimum hybridization conditions. The specific hybridization of amplicons to the polynucleotide probes may be read out with a reader after washing off nucleic acids that are not bound. For example, the detection of a specific hybridization of a biotinylated amplicon with the immobilized probes may be carried out using streptavidin-horseradish peroxidase conjugate. The color precipitates formed by enzymatic conversion of the added substrate tetramethylbenzidine (TMB) at the individual spots are detected by means of an analytic device and read out. The technology of microarrays is generally known to the skilled person. Probe systems such as those that may be used in principle for the detection of the amplicons obtained in the PCR of the invention are commercially available, e.g. under the trade name AT® System (Clondiag Chip Technologies, 07749 Jena, Germany). The skilled person, due to his technical knowledge, is easily enabled to develop microarrays adapted to the particular detection of micro-organisms.

In the case of qPCR, detection of the amplicons takes place during the individual amplification cycles. For example, the amplicons may be detected using dyes that bind to double-stranded DNA. When stimulated by a suitable wavelength, these dyes show a higher fluorescence intensity when bound to double-stranded DNA. Detection by means of dyes binding to double strands is described, e.g., in EP-A-0 512 334. In accordance with a preferred embodiment, the amplicons are detected using fluorescence-labelled hybridization probes which emit fluorescence signals only when they are bound to the target nucleic acid. Examples of probes which may be used for the detection in qPCR are known to the skilled person and include, e.g., TaqMan™ probes (see, e.g., EP-A-0 543 942 and U.S. Pat. No. 5,210,015), Molecular Beacons (see U.S. Pat. No. 5,118,801), Scorpion-Primers, Lux® primers and FRET probes (see WO 97/46707, WO 97/46712 and WO 97/46714). FRET probes—as described in the indicated literature—may also be used for melting curve analysis.

Both non-quantitative and quantitative amplification may be followed by sequencing for further differentiation.

The methods and means of the invention may be employed in various fields and may generally be used for determining bacteria and/or fungi and/or resistances thereof. The method of the invention is suitable for testing of any desired sample material in which bacteria and fungi, in particular pathogenic bacteria and fungi, may occur, e.g. an environmental sample, a food sample, or a biological sample, e.g., a clinical sample. The biological sample may be a plant sample; in a typical case the biological or clinical sample is, however, a human or animal sample, in particular a sample from a mammal. Typically, the sample is a human or a animal tissue sample, e.g. a biopsy or a body fluid or a product derived therefrom. In a preferred manner, the sample is a body fluid or a product derived therefrom, e.g. blood or a blood product, such as full blood, serum, plasma, thrombocyte concentrate, cerebro-spinal fluid, liquor, urine and pleural, ascites, pericardial, peritoneal or synovial fluid. In accordance with a preferred embodiment, the methods and means of the invention may be used for detecting pathogenic bacteria, fungi and/or antibiotic and antimycotic resistances in clinical samples. According to an advantageous embodiment, the methods and means of the invention may be used for detecting of contaminations in thrombocyte concentrates. According to a further preferred embodiment, the methods and means of the invention may be used for detection and early diagnosis of pathogens and/or resistances of inflammatory diseases involving undetected infection (also referred to as “Systemic Inflammatory Response Syndrome”, SIRS, according to the criteria of the consensus conference of the American College of Chest Physicians/Society of Critical Care Medicine Consensus Conference, ACCP/SCCM”, Crit. Care Med. 1992; 274:968-974). According to another advantageous embodiment, the methods and means of the invention may be used for detection and early diagnosis of infectious diseases, in particular systemic infections. According to another, quite particularly preferred embodiment, the methods and means of the invention may be used for detection and early diagnosis of sepsis. In accordance with one further preferred embodiment, the methods and means of the invention may be used for detection and early diagnosis of spontaneous bacterial peritonitis. According to another preferred embodiment, the methods and means of the invention may be used for detection and early diagnosis of endocarditis. In all of these cases the primers are preferably selected such that they hybridize to nucleic acid sequences of the micro-organisms or resistances expected in the sample material and allow the amplification thereof. Thus, e.g., primers which specifically hybridize to nucleic acid sequences of the micro-organisms shown in FIG. 3A are preferably used for early diagnosis of sepsis.

In summary, the present invention thus relates to methods and means for determining pathogenic fungi in a sample material, e.g. blood. In the method, the bacterial DNA is initially enriched from the total DNA of the sample material, and then the enriched DNA is amplified with specific primer pairs. The detection of the obtained amplicons allows the accurate identification of bacteria and fungi contained in the sample material and of their resistances. The methods and means of the invention are characterized in that they allow a simple and rapid determination of bacteria and fungi in sample materials. It was surprisingly found that the simple combination of a specific enrichment step for bacterial and fungal DNA in relation to other DNA in the sample material, in particular human DNA, and of an amplification step allows not only to considerably enhance the detection sensitivity for individual bacteria and fungi, but that it is even possible in this way to detect various different genera and species of bacteria and fungi in parallel with a high sensitivity. This allows a simultaneous, rapid and reliable determination of pathogens and allows the attending physician to optimally adapt the therapy to the detected pathogens without loss of time. The method of the invention thus represents an important aid in the physician's decision concerning the appropriate therapeutic treatment. As the method of the invention is performed with primers that allow the general detection of bacteria and/or fungi, an infection may moreover be detected even if the pathogens are bacteria and fungi that hitherto occurred rarely or not yet at all.

### BRIEF DESCRIPTION OF FIGURES

The present invention shall be described in more detail by way of the following examples and figures.

FIG. 1 shows a graphic representation of the dependence of enrichment of prokaryotic and fungal DNA on the initial content of total DNA;

FIG. 2 shows an agarose gel electrophoresis for the detection of E. coli in ascites fluid by means of multiplex PCR following enrichment of prokaryotic and fungal DNA;

FIG. 3 shows the detection of bacterial DNA following DNA enrichment and multiplex PCR with 50 different primer pairs from a blood sample to which the DNA of various micro-organisms had been added; FIG. 3A shows the list of micro-organisms against which the employed primers were directed; FIG. 3B shows a photograph of an agarose gel with PCR amplicons of the spiked micro-organisms; FIG. 3C shows the sample allocation of the gel of FIG. 3B;

FIG. 4 shows the spotting scheme for the probes of a microarray for an exemplary, probe-based detection of biotinylated PCR amplicons that are specific for particular bacteria, fungi and resistances;

FIG. 5 shows a photographic image of the microarray of FIG. 4 following hybridization with the biotinylated, E. coli-specific PCR amplicon irp2;

FIG. 6 shows an agarose gel electrophoresis of a multiplex PCR of mechanically lyzed full blood samples of healthy donors to which overnight cultures of C. albicans and S. pyogenes were added;

FIG. 7 shows a qPCR evaluation of a mechanically lyzed full blood sample of a healthy donor to which an overnight culture of C. albicans was added; and

FIG. 8 shows a qPCR evaluation of a mechanically lyzed full blood sample of a healthy donor to which an overnight culture of S. pyogenes was added.

### EXAMPLES

The following examples merely represent working examples of the present invention and are not intended to limit the scope of the invention in any way.

### Example 1

### Detection of Pathogens of Spontaneous Bacterial Peritonitis (SBP)

### Sampling

The samples originated from the Klinik für lnnere Medizin, Department for Gastroenterology, Hepatology and Infectiology of Friedrich-Schiller-Universität Jena, Germany. Following approval by the local ethics commission concerning the projected study, ascites fluid was taken from 75 patients with suspected SBP. As the gold standard the total cell count was measured, and in cases where the number exceeded 250 cells/μl, the number of neutrophil cells was determined. Ascites cultures were prepared in blood culture bottles (aerobic/anaerobic) inoculated with 5 ml of ascites. Total DNA was determined following extraction with a Nanodrop® apparatus. A sub-group of 14 patients (6 females (average age 67 years), 8 males (average age 57.6 years) was selected in which the number of neutrophil cells exceeded the threshold value of the gold standard, or the ascites culture was positive, or other indications (e.g. by way of a blood culture) pointed to a systemic infection, or the 16S-rDNA-qPCR carried out in a second step as described below resulted in significantly increased copy numbers of the target sequence.

For sample processing for the nucleic acid test (NAT), 50 ml of ascites was placed in 50 ml-Falcon tubes. The cells were counted and centrifuged. The pellet was resuspended in 5 ml of the remaining supernatant and stored at −80° C.

Isolation of Total DNA from Ascites Samples

The isolation of total DNA was carried out using LOOXSTER® in accordance with the manufacturer's specifications.

### Cell Lysis

100 μl of lysozyme solution was added to the thawed ascites sample (final concentration 1 mg/ml), and following brief vortexing, incubation was performed for 1 h at 37° C. 5 ml of lysis buffer A and 100 μl of protease solution were added, and following brief vortexing, was performed during 1 h at 50° C. The sample was vortexed for 20 s and applied to the membrane of a 50-ml tube.

### Binding and Washing

The tube was centrifuged during 2 min at 3,000×g. The tube was changed, 5 ml of buffer B was added, and centrifuging was performed once more. Again 5 ml of buffer B was added, and centrifuging was performed once more.

### Elution

The tube was changed, 2.5 ml of buffer C was applied to the membrane, and incubation was performed for 2 min at room temperature. The tube was subjected to centrifugation for 1 min at 3,000×g, 2.5 ml of buffer C was additionally applied to the membrane, and the tube was subjected once more to centrifugation. The membrane insert was discarded, and the eluate transferred to a fresh 15 ml tube.

### Precipitation

4 ml of isopropanol was added, followed by careful mixing and centrifugation for 60 min at 3,000×g. The supernatant was removed, and the pellet was washed with 2 ml of ice-cold 70-% ethanol. The tube was subjected to centrifugation for 5 min at 3,000×g, and the pellet was dried at room temperature. The DNA was dissolved in 200 μl of distilled water (DNA- and DNase-free) at 50° C. for 1 h. 16 μl was taken for qPCR analysis prior to enrichment of prokaryotic and fungal DNA. The remaining 184 μl was mixed into 184 μl of 2× buffer D.

### Enrichment of Prokaryotic and Fungal DNA

The enrichment of specific genomic, bacterial and fungal DNA was performed with the LOOXSTER® Kit in accordance with the manufacturer's specifications. The kit contains columns, collecting tubes and reagents for the enrichment of prokaryotic DNA from samples with mixed DNA from human and bacterial DNA that are also suited for the enrichment of fungal DNA. The experimental arrangement is summarized below.

### Binding

The columns were conditioned in accordance with specifications in LOOXSTER®. 368 μl of the DNA dissolved in buffer D was added to the prepared column. The mixture of matrix/DNA was carefully pipetted up and down and incubated for 30 min at room temperature. The column was centrifuged at room temperature for 30 s at 1,000×g, and the flow-through was discarded.

### Washing

2×300 μl of buffer D was added to the column followed by two times centrifugation at room temperature for 30 s at 1,000×g.

### Elution Step

The column was transferred into a new 2-ml tube, and 300 μl of buffer D was added. The mixture of matrix/DNA was carefully pipetted up and down. The column was incubated for 5 min at room temperature and centrifuged at room temperature for 30 s at 1,000×g. 300 μl of buffer E was again added to the column, followed by centrifugation for 30 s at 1,000×g. The volume of the eluate was 600 μl.

### Precipitation

The eluated DNA was precipitated by adding 5 μl of Solution G, 60 μl of NaAc, pH 5.2, and 480 μl of isopropanol. Following brief vortexing (10 s), the sample was centrifuged at 4° C. for 60 min at 16,000×g, and the supernatant was discarded. The pellet was washed 2× with 1 ml of ice-cold 70-% ethanol, centrifuged for 5 min at 16,000×g, and the supernatant was discarded. The pellet was dried at room temperature and dissolved in 30 μl of DNA- and DNase-free water at 50° C. for 1 h. The DNA concentration was determined with the aid of a Nanodrop® apparatus.

Real-Time PCR with 16S Primers

Quantification of prokaryotic DNA was carried out by means of 16S rDNA qPCR. The total DNA concentration was adjusted to an optimum concentration of 200 ng/reaction. Although the content of isolated DNA was low, identical concentrations of these relevant samples with and without enrichment were examined by the LOOXSTER® system. A negative control with DNA-free water was run analogously to the patients' samples in order to determine a threshold (cut-off) for the handling of bacterial DNA. Bacterial DNA (10⁵ genome copies) was added to an aliquot of the sample of each single patient in order to ascertain a potential inhibition of the PCR. Controls without template (NTC) were also included. The detection was based on fluorescence as a result of incorporation of SYBR® Green in double-strand DNA. 25 μl of reaction volume consisted of ≦200 ng of total genome DNA in 10 μl, 12.5 μl 2× QuantiTect® SYBR® Green PCR Master Mix (QIAGEN®), and 1.25 μl (10 pmol final concentration) of forward and reverse primer each. All steps were performed in duplicate using a Rotor-Gene RG-3000 qPCR apparatus (Corbett Life Science, Sydney, Australia). DNA denaturation at the beginning was performed for 15 min at 94° C., followed by 45 cycles of 94° C. for 30 s, 50° C. for 30 s, 72° C. for 1 min. Calculation was performed using the Rotor-Gene 6 software.

### Multiplex PCR

Identification of the bacterial and fungal pathogens for an optimum therapeutic approach took place by means of non-quantitative multiplex PCR. The reaction was carried out in two reaction vessels having two primer pools (primer pools I and II) containing primer pairs with nucleic acid sequences that were specific for bacteria and fungi in general as well as for particular bacteria and fungus genera, particular bacteria species, as well as selected resistances. The following table provides an overview of the bacteria, fungi and resistances covered in this test.

TABLE 1
Tested bacteria, fungi and resistances

Bacteria:
Burkholderia cepacia²
Enterobacter aerogenes²
E. cloacae¹
E. faecium²
E. faecalis¹
Escherichia coli^{1,2}
Klebsiella oxytoca¹
Klebsiella pneumoniae²
Morganella morganii¹
Prevotella spp²
P. melanogenica^{#}
Proteus mirabilis¹
Pseudomonas aeruginosa²
Staphylococcus spp²
Staphylococcus aureus¹
Staphylococcus haemolyticus*
Stenotrophomonas maltophila¹
Streptococci of the Viridans group
S. pneumoniae¹
S. pyogenes¹
Fungi:
Fungi spp²
Aspergillus fumigatus¹
Candida albicans
Resistances:
Methiciliin²
¹Primer pool I;
²Primer pool II;
*Tested species were detected with the Staphylococcus spp primer (primer pool II)
^{#}tested species were detected with the Prevotella spp primer (primer pool II)

The DNA concentration was adjusted to an optimum concentration of ≦500 ng/reaction. If the isolated DNA content was not sufficient, lower concentrations were used for the LOOXSTER® treatment. The reaction volumes of 25 μl consisted of a variable quantity of template DNA, 12.5 μl of 2× Multiplex PCR Master Mix (Quiagen®), 2.5 μl of Primer Mix (10 μmol final concentration, used as primer pools I and II) and DNA- and DNase-free water. An initial DNA denaturation was carried out for 15 min at 95° C. for activating of HotStar Tag® DNA polymerase (Quiagen®), followed by 30 cycles of 94° C. for 30 s, 59° C. for 1.5 min, and 72° C. for 45 s. The program ended with a terminal hybridization step of 72° C. for 10 min. All of the steps were carried out with a Mastercycler® Gradient S (Eppendorf AG, Hamburg, Germany). The samples were analyzed on a 2-% agarose gel.

### Results

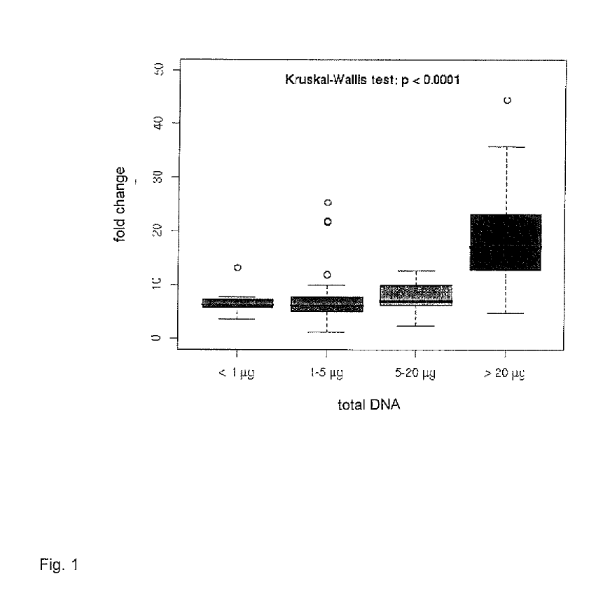

The data obtained in the multiplex PCR were compared to the gold standard (increased number of polymorphic cells in the ascites ≧250/μl) and those of the ascites cultures. The efficiency of the LOOXSTER® method in dependence on the DNA content applied to the column was determined by means of 16S-rDNA PCR before and after LOOXSTER® (FIG. 1). As shown in FIG. 1, the concentration factor for the enrichment of prokaryotic and fungal DNA increases with higher DNA quantities. From full blood it is possible to isolate more than 20 μg of DNA. In ascites fluids having variable DNA concentrations of from <1 to >20 μg, a significant enrichment was observed in each case.

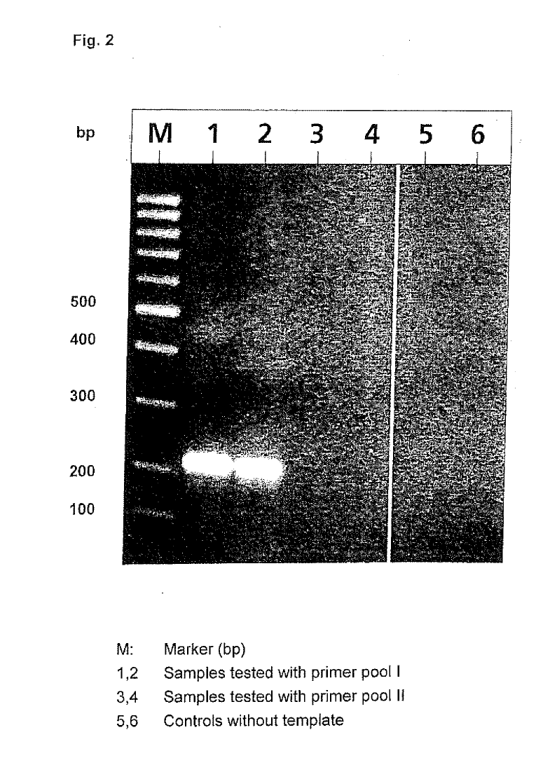

In 19 samples originating from the above-mentioned sub-group of 14 patients with suspected SBP, the multiplex PCR was positive in all cases in which the ascites cultures were positive and in which an increased number of neutrophil cells was counted. It was furthermore detected that a patient had a multiple infection with E. faecalis, E. coli and E. faecium, where the ascites culture and also the total cell count were negative (<250 cells/μl). Table 2 summarizes the results of the study on the 19 samples, and Table 3 represents a selection of the case reports for patients for which SBP was confirmed by the method of the invention used in the study. FIG. 2 shows the gel electrophoresis of a multiplex PCR on an agarose gel for the detection of an E. coli infection in two samples of ascites fluid taken from patient 1 within 2 days. Lanes 1 and 2 show samples tested with primer pool I. The bands at 218 by are specific for E. coli amplicons.

TABLE 2
Statistics of the ascites study
Polymorphous neutrophil cells

| | | Negative | positive | NPV/PPV |
|---|---|---|---|---|
| Multiplex PCR | negative | 15 | 0 | 100% |
| | positive | 1 | 3 | 75% |
| | spec./sens. | 93.75% | 100% | |

NPV/PPV: predicted negative/positive value
spec./sens.: Specificity and sensitivity of multiplex PCR compared with gold standard

TABLE 3
Selected case reports from the sub-group including 14 patients

| | | | | qPCR | | | |
|---|---|---|---|---|---|---|---|
| | | Total | Number of | 16S | | Blood | Note |
| | Ascites | cell | neutrophilic | rDNA | Multiplex | culture | (diagnosis; |
| Patient | culture¹ | count | cells | copies | PCR | (optional) | antibiosis |
| Threshold | — | ≧250² | ≧250³ | ≧50% | — | — | |
| | | | | above | | | |
| | | | | average 4 | | | |
| 1 | E. coli, | 9,700 | 6,220 | yes | E. coli | negative | Sepsis; |
| | S. haemolyticus | | | | | | cont. |
| | | | | | | | therapy |
| | | | | | | | with |
| | | | | | | | Ceftazidim |
| 2 | negative | 90 | — | yes | E. faecalis, | E. faecalis | pyic |
| | | | | | E. coli, | | peritonitis; |
| | | | | | E. faecium | | therapy |
| | | | | | | | with |
| | | | | | | | Ceftriaxon/ |
| | | | | | | | Metronidazol; |
| | | | | | | | after |
| | | | | | | | pathogen |
| | | | | | | | detection |
| | | | | | | | in culture, |
| | | | | | | | change to |
| | | | | | | | Tazobac |
| 3 | E. coli | 740 | 390 | no | E. coli | E. coli | Sepsis; |
| | | | | | | | cont. |
| | | | | | | | therapy |
| | | | | | | | with |
| | | | | | | | Ciprofloxacin |

¹Pathogens in cases of positive ascites cultures specified by culture methods
²Threshold value for the determination of the number of neutrophilic cells
³Gold standard threshold for SBP diagnosis
⁴ qPCR cut-off

The nucleic acid-based PCR method gave positive results in all three selected cases. The total cell count and the number of neutrophil cells were increased, and the ascites cultures were positive in two cases. In addition, the multiplex PCR revealed a multiple infection in one case (Patient 2) in which neither the cell count nor the number of copies determined by means of qPCR had been elevated. The patient was initially treated with Ceftriaxon/Metronidazol. Three days after taking blood and ascites fluid, the blood culture was positive for E. faecalis, while the parallel ascites cultures remained negative. Accordingly the therapy was changed to Tazobac which does not inhibit the growth of E. faecium. In addition, E. coli, E. faecalis and E. faecium were found in wound smears. The same three organisms (E. coli, E. faecalis and E. faecium) were, however, also found with multiplex PCR. This shows that within about 6 h, multiplex PCR procures the same results as blood and ascites cultures within several days. The use of a multiplex PCR in combination with an enrichment of bacterial and fungal DNA from total DNA thus allows rapid and early pathogen detection as well as an appropriate and early antibiotic therapy.

Accordingly, in this case neither the current gold standard method nor a 16S rDNA qPCR is sufficient by itself for diagnosing a SBP, not to mention the fact that these methods do not result in any information concerning a specific antibiotic treatment.

### Example 2

### Detection of Bacteria and Fungi in Spiked Samples by Means of PCR and Gel Electrophoresis

A blood sample was spiked with bacterial DNA of S. aureus, E. coli and K. pneumoniae. Total DNA preparation and enrichment of bacteria DNA with LOOXSTER® was carried out as described in Example 1.

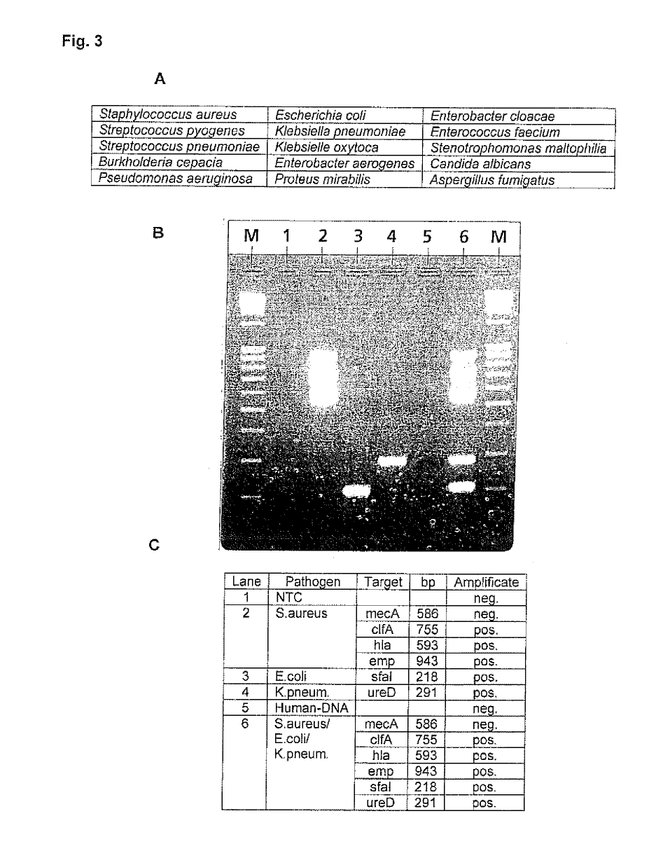

The obtained DNA samples were amplified with 50 sepsis-specific primer pairs that were specific for particular nucleic acid sequences of the bacteria and fungi shown in FIG. 3A, in different batches by means of non-quantitative multiplex PCR as described in Example 1. The samples were analyzed on a 2-% agarose gel. FIG. 3B shows a corresponding agarose gel with PCR amplicons of the added bacterial DNA. FIG. 3C shows the associated sample application on the gel and the expected amplicon sizes for the selected PCR targets (M is Marker).

The test shows that the three bacteria species S. aureus, E. coli and K. pneumoniae could be detected specifically following DNA enrichment and multiplex PCR with specific primer pairs.

### Example 3

### Multiplex PCR and Probe-Based Detection of Escherichia coli in Spiked Samples

A blood sample was spiked with bacterial DNA of E. coli as described in Example 2. Total DNA preparation and enrichment of bacterial DNA with LOOXSTER® were carried out as described in Example 1. For a detection of E. coli a multiplex PCR in the presence of biotin-16 dUTP with primers directed to the gene irp2 was carried out.

The successful incorporation of the labelled nucleotide was detected by Southern-Blotting. For blotting, two layers of Whatman filter paper and a nitrocellulose membrane were soaked in 0.5×TBE buffer and successively placed on the anode (−). Then the gel was placed on the membrane and covered with two layers of Whatman filter paper soaked in 0.5×TBE. Finally, the cathode (+) was applied and the apparatus was connected at 2 A during 12 min. UV crosslinking was employed for fixation. The membrane was irradiated with UV light for 1 min at 150 mJ/cm² and afterwards dried at the air for 30 min. Subsequently the membrane was blocked with blocking buffer for 1 h at room temperature. Then the membrane was washed three times for 5 min with TBST buffer. The membrane was treated with streptavidin HRP diluted 1:2000 with blocking solution followed by 30 minutes of incubation at room temperature. This resulted in the formation of a typical streptavidin-biotin conjugate. Afterwards the membrane was washed 3 times for 5 min with TBST buffer, and the substrate was placed on the membrane. TMB was used as a substrate. Developing the blot took 10 min. Blue dye formed at those places where the biotin had been incorporated (not shown). The expected 200 bp-irp2 amplicon was also detected by gel electrophoresis (data not shown).

The presence of the biotinylated 200 bp-irp2 amplicon was subsequently detected as follows by a probe-based assay (microarray).

### Probe Design and Chip Production

For all of the selected oligonucleotides, care was taken that at least 7-8 base pair mismatches (temperature difference 14-16° C.) to all other DNA sequences deposited in the NCBI-GenBank (m, est human) were present. The sequences were calculated with the program Arraydesigner® under the following specifications:
probe length 35±5 bases
melting temperature approx. 70° C.
balanced GC content (A/T:G/C=1:1)
2 non-overlapping probes per target for the specific pathogen detection
avoidance of cross-reactions with human and other bacterial targets
poly-T (10 T's) at the 3′ end of the probes for mobility of the probes on the array
amino-modification at the 3′ end of the oligonucleotides for coupling to the surface of the DNA microarray

Cross-reactions were excluded with defined primers of all of the used targets by computer-based matching of the probe against all of the primers/amplicons of the employed targets.

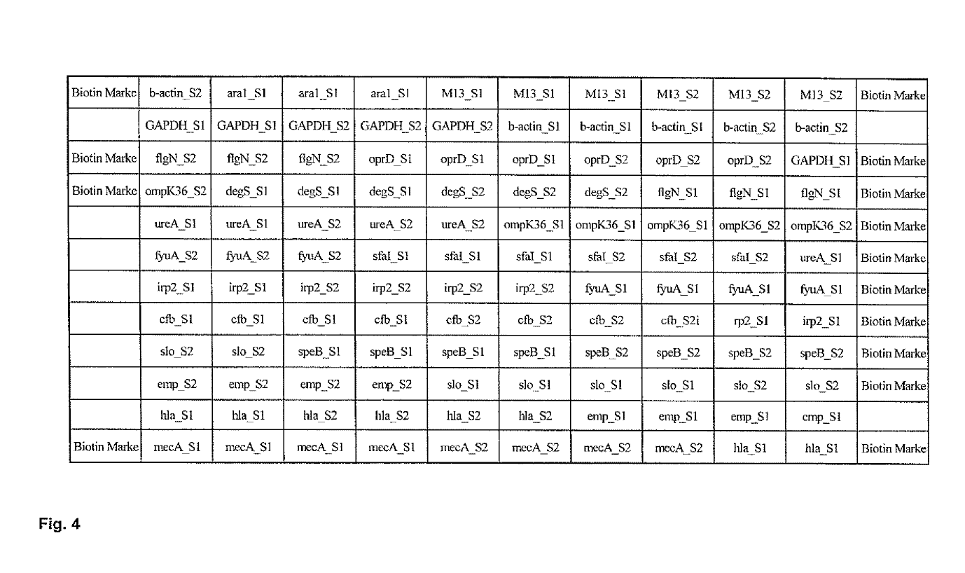

Detection of the PCR fragments was based on the AT® system (Clondiag Chip Technologies, 07749 Jena, Germany). Preparation of array tubes was performed at Clondiag in accordance with the spotting plan shown in FIG. 4 which shows the arrangement of the individual oligonucleotides on the DNA microarray. In addition, biotin probes were immobilized on the marginal area of the array (biotin Marker). These serve as a positive control, for owing to the reaction of the biotin with the streptavidin used for detection, formation of a spot will always occur at these probes. Moreover the intensity of the biotin probes allows statements concerning the ratio of sample quantity to gene probes present. The intensity of the spots generated by the specific gene probes should not exceed the intensity of the biotin probes, for this indicates overloading of the array with the PCR fragments and may lead to false-positive results.

### Hybridization

Biotinylated amplicons were used directly for hybridization. To this end, 4 μl of the biotinylated PCR product was taken up in 96 μl of hybridization buffer and denaturated outside the Array-Tubes® for 5 min at 95° C., and then immediately cooled on ice for 120 s.

The Array-Tubes® were pre-washed twice. All of the used solutions were carefully removed with a plastic Pasteur pipette after the end of the reaction period. Addition of 500 μl of Aqua bidest resulted in denaturation during 5 min at 50° C. and 550 rpm on the thermomixer. Then 500 μl of hybridization buffer was added and incubation was carried out for 5 min at 50° C. and 550 rpm. After this, 100 μl of the denaturated sample was subjected to hybridization for 60 min at 50° C. and 550 rpm in the AT® system. After three washing steps, firstly with 500 μl of washing solution 1 (5 min at 40° C. and 550 rpm), secondly with 500 μl of washing solution 2 (5 min at 30° C. and 550 rpm) and finally with 500 μl of washing solution 3 (5 min at 30° C. and 550 rpm), 100 μl of a freshly prepared 2-% blocking buffer was placed on the array for 15′ at 30° C. and 550 rpm in order to damp its background signal. Of the freshly produced streptavidin-HRP conjugate solution, 100 μl was then pipetted onto the array and subjected to conjugation for 15 min at 30° C. and 550 rpm. After this, washing with 500 μl of washing solution 1 (5 min at 30° C. and 550 rpm) was performed. The second washing step was carried out by adding 500 μl of washing solution 2 during 5 min at 20° C. and 550 rpm. Finally, 500 μl of washing solution 3 was added to the AT®, and incubation was performed during 5 min at 20° C. and 550 rpm. The Array-Tube® was inserted in the temperature-controlled reading tray (25° C.) of the AT® reader in which the last washing solution was removed under visual control via the CCD camera of the reader and the camera of the reader was focused. Immediately after this, detection was performed by the addition of 100 μl of peroxidase substrate (TMB). 60 images were taken, i.e., one image every 10 seconds. The CCD camera measures the transmission of white light through the Array-Tubes®. The data thus obtained were evaluated with the IconoClust® software.

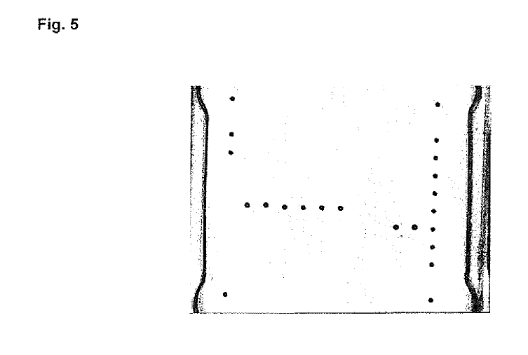

FIG. 5 represents a photographic image of the hybridization result of the single assay of the biotinylated irp2 of E. coli. It was found that the 200 by amplicons of the irp2 gene could be detected without cross-reactions with other probes.

### Example 4

### Non-Quantitative and Quantitative PCR for the Detection of Streptococcus pyogenes and Candida albicans in Full Blood Samples

Overnight cultures (10⁵ cells) of C. albicans [ATCC MYA-2876] and S. pyogenes [Varig 42440 (Institut für Medizinische Mikrobioiogie, Jena), positive blood culture of a septic] were added to full blood samples of healthy donors. Following addition of 2 g of glass beads (G8772 glass beads, acid-washed, 425-600 μm, Sigma Aldrich Chemie GmbH, Schellendorf, Germany) and 100 μl of protease, the cells were lyzed mechanically by 2×2 min of vortexing, in each instance followed by 2-minute incubation at 50° C. The isolation of total DNA was carried out with the Genomic Maxi AX Blood-Kit (A&A Biotechnology, Gdynia, Poland) and the enrichment of bacterial and fungal DNA was carried out with LOOXSTER® as described in Example 1.

### Non-Quantitative PCR

Identification of C. albicans end S. pyogenes was carried out by non-quantitative multiplex PCR. The DNA concentration of the LOOXSTER® eluates in the multiplex PCR batch was adjusted to 500 ng (NanoDrop® DNA concentration determinations). The reaction volumes of 25 μl (two primer pools with several species-specific primer pairs, i.e., two reaction batches per sample) consisted of 5 μl of template DNA, 5 μl of DNA-free water for cell culture (FAA), 12.5 μl of 2× Multiplex PCR Master Mix (QIAGEN®, Hilden, Germany) and 2.5 μl of 10× Primer Mix (10 μmol final concentration). An initial denaturation at 95° C. during 15 min was required for the activation of the HotStarTaq® DNA-Polymerase (QIAGEN®). The entire PCR thermocycler program can be seen in Table 4 below.

TABLE 4
Thermocycler program

| | Partial | Temperature | Time | |
|---|---|---|---|---|
| Main section | section | [° C.] | [s] | Cycles |
| initial | | 95 | 900 | 1 |
| denaturation | | | | |
| Amplification | Denaturation | 94 | 45 | |
| | Annealing | 59 | 30 | 30 |
| | Extension | 72 | 45 | |
| Final extension | | 72 | 600 | 1 |

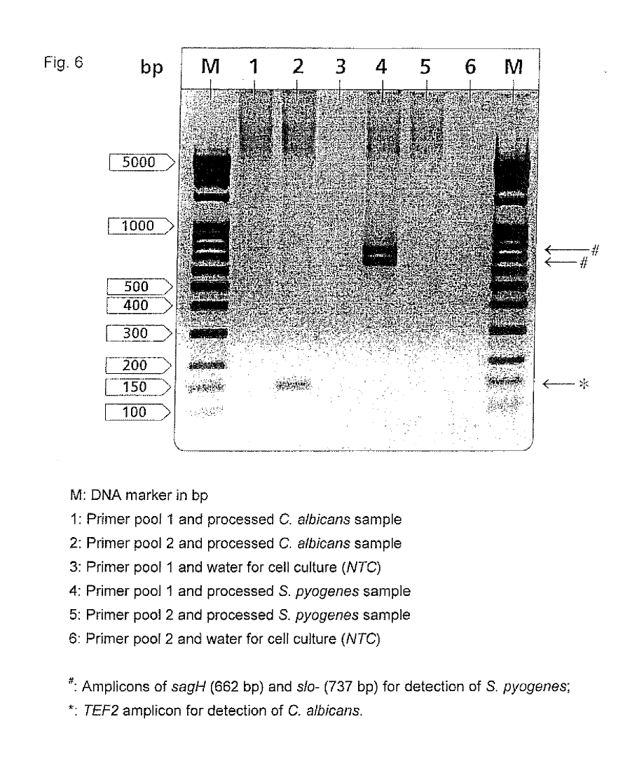

All incubation steps were carried out on a Mastercycler® ep Gradient S (Eppendorf AG, Hamburg). The PCR products were separated on 1.5-% agarose gels. The results are represented in FIG. 6 which shows a photograph of the corresponding agarose gel: M: DNA marker (indication in bp), 1: primer pool 1 and processed C. albicans blood sample, 2: primer pool 2 and processed C. albicans blood sample, 3: primer pool 1 and water for cell culture (NTC), 4: primer pool 1 and processed S. pyogenes blood sample, 5: primer pool 2 and processed S. pyogenes blood sample, 6: primer pool 2 and water for cell culture (NTC). Lane 4 shows the amplicons of sages (662 bp) and slo− (737 bp) for the detection of S. pyogenes (#), and lane 2 shows the TEF2 amplicon for the detection of C. albicans (*).

The results show that C. albicans and S. pyogenes can specifically be detected in blood samples by the method of the invention.

Real-Time PCR (qPCR) Following Mechanical Cell Lysis

Quantification of fungal and bacterial targets was carried out by quantitative PCR (qPCR and real-time PCR, respectively) using 18S rDNA- and gene-specific primers. The total DNA quantity was 200 ng/reaction (on the basis of Nanoprop® DNA measurements). DNA enriched as described above with LOOXSTER® was employed. As a negative control (for determining the threshold value (Threshold) or “cut-off” for pathogen DNA), DNA-free water for cell culture (PAA) was employed. Detection is based on the intercalation of the fluorescent dye SYBR® Green in DNA. The 25-μl reaction batch consisted of 10 μl of genomic DNA (200 ng), 12.5 μl of 2× QuantiTect® SYBR® Green PCR Master Mix (QIAGEN®) and 1.25 μl (10 μmol final concentration) of forward and reverse primer each. For the detection of C. albicans, the 18S-rDNA primer pair panfneu11/12 was employed, and for S. pyogenes the gene-specific primer pair sagA.

All reaction steps were carried out in two parallels in a Rotor-Gene™ RG 3000 (Corbett Life Science, Sydney, Australia). The thermocycler program is shown in Table 5 below. Evaluation took place using the system-compatible Rotor-Gene 6 software.

TABLE 5
qPCR Thermocycler program

| | Partial | Temperature | Time | |
|---|---|---|---|---|
| Main section | section | [° C.] | [s] | Cycles |
| Initial | | 94 | 900 | 1 |
| denaturation | | | | |
| | Denaturation | 94 | 30 | |
| Amplification | Annealing | 55 | 30 | 45 |
| | Extension | 72 | 60 | |
| Melting curve | | 50-95 | 30 in Step 1, | 1 |
| analysis | | | 5 in the | |
| | | | following steps | |
| | | | (1 degree/step) | |

### Results

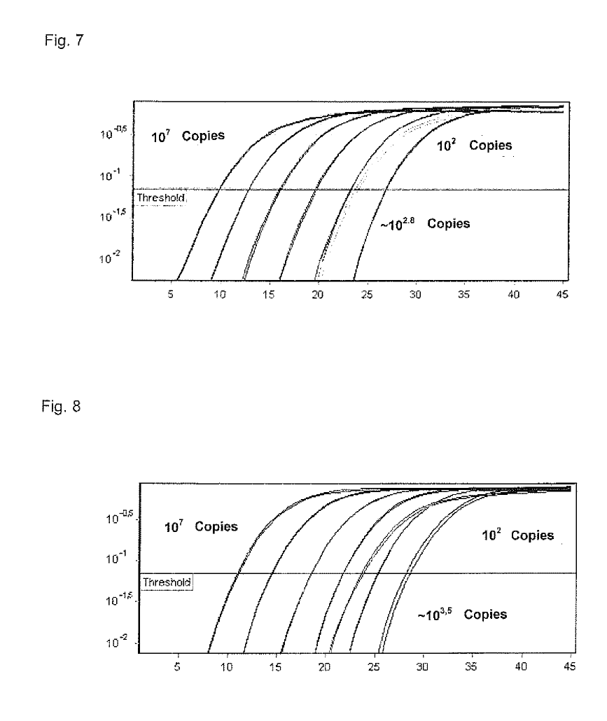

FIGS. 7 and 8 show the results following Rotor-Gene 6 evaluation. Relative fluorescence values (ordinate) were plotted over PCR cycles (abcissa). The basis used for calculation was the determination of the Cₜ value, i.e., the cycle number at which the fluorescence threshold value (“Threshold”) is exceeded for the first time within one amplicon-specific fluorescence curve.

FIG. 7 shows the qPCR evaluation of the mechanically lyzed full blood sample of a healthy donor to which an overnight culture of C. albicans (ATCC MYA-2876) had been added. The relative fluorescence was plotted over the PCR cycle number. What is represented is the C. albicans standard series (black) of from 10⁷ to 10² copies. The retrieval rate of the spiked cell count of 10⁵ of the mechanically processed C. albicans sample is about 14% (˜10^{2.9} copies corresponds to ˜490 pg of fungal DNA at 35 fg per genome copy).

FIG. 8 shows the qPCR evaluation of the mechanically lyzed full blood sample of a healthy donor to which an overnight culture of S. pyogenes [Varia 42440 (Institut für Medizinische Mikrobiologie, Jena), positive blood culture of a septic] had been added. The relative fluorescence was plotted over the PCR cycle number. What is represented is the S. pyogenes standard series (black) of from 10⁷ to 10² copies. The retrieval rate of the spiked cell count amounting to 10⁵ of the mechanically processed S. pyogenes sample is about 56% (˜10^{3.5} copies corresponds to ˜112 pg bacterial DNA at 2 fg per genome copy).

The results show that bacteria and fungi in blood samples, following enrichment of their DNA by proteins which specifically bind the bacterial and fungal DNA, can be detected by means of qPCR, wherein the qPCR moreover allows a statement concerning the concentration of the pathogens in the blood sample.

1.-28. (canceled)

29. A method for determining bacteria and fungi and/or resistances thereof in a sample material for detecting infections and supporting a therapy decision or for detecting contaminations, wherein said method comprises the following steps:
a) enriching bacterial and fungal DNA from total DNA of a human or animal sample material selected from tissue samples, body fluids and products derived therefrom;
b) multiplexing amplification of the enriched bacterial and fungal DNA obtained in step a) using at least 20 different primer pairs selected from primer pairs of at least two of groups (i) to (vii), wherein:
group (i) comprises at least one primer pair which is suited for the specific amplification of a region of a particular nucleic acid sequence that is specific for a plurality of bacteria families;
group (ii) comprises at least one primer pair which is suited for the specific amplification of a region of a particular nucleic acid sequence that is specific for a plurality of fungus families;
group (iii) comprises at least one primer pair which is suited for the specific amplification of a region of a particular nucleic acid sequence that is specific for a selected bacteria genus;
group (iv) comprises at least one primer pair which is suited for the specific amplification of a region of a particular nucleic acid sequence that is specific for a selected bacteria species;
group (v) comprises at least one primer pair which is suited for the specific amplification of a region of a particular nucleic acid sequence that is specific for the expression of a selected antibiotics or antimycotics resistance;
group (vi) comprises at least one primer pair which is suited for the specific amplification of a region of a particular nucleic acid sequence that is specific for a selected fungus genus; and
group (vii) comprises at least one primer pair for the specific amplification of a region of a particular nucleic acid sequence that is specific for a selected fungus species; and
c) detecting the presence of amplicons formed in step b),
wherein the presence of amplicons formed with the at least one primer pair of group (i) indicates the presence of bacteria, the presence of amplicons formed with the at least one primer pair of group (ii) indicates the presence of fungi, the presence of amplicons formed with the at least one primer pair of group (iii) indicates the presence of the selected bacteria genus; the presence of amplicons formed with the at least one primer pair of group (iv) indicates the presence of the selected bacteria species; the presence of amplicons formed with the at least one primer pair of group (v) indicates the presence of the selected antibiotic or antimycotic resistance; the presence of amplicons formed with the at least one primer pair of group (vi) indicates the presence of the selected fungus genus; and the presence of amplicons formed with the at least one primer pair of group (vii) indicates the presence of the selected fungus species.

30. The method of claim 29, wherein the sample material is a body fluid or a product derived therefrom, in particular blood or a blood product, selected from the group consisting of full blood, plasma, serum, thrombocyte concentrate, cerebro-spinal fluid, liquor, urine, pleural fluid, ascites fluid, pericardial fluid, peritoneal fluid and synovial fluid.

31. The method of claim 29, wherein the enrichment of the bacterial and/or fungal DNA is carried out by contacting the total DNA obtained from the sample material with a protein or a polypeptide capable of binding to non-methylated CpG motifs.

32. The method of claim 29, wherein the at least one primer pair of group (i) is a primer pair which specifically hybridizes to the nucleic acid sequence of the gene for bacterial 16S rDNA.

33. The method of claim 29, wherein the at least one primer pair of group (ii) is a primer pair which specifically hybridizes to the nucleic acid sequence of the gene for fungal 18S rDNA.

34. The method of claim 29, wherein the amplification in step b) is performed under conditions under which the amplicons are labelled with a detectable marker.

35. The method of claim 29, wherein the amplification in step b) is performed by means of non-quantitative PCR.

36. The method of claim 29, wherein the amplification in step b) is performed by means of real-time quantitative PCR (qPCR).

37. The method of claim 29, wherein the detection of the amplicons obtained in step b) is performed by means of gel electrophoresis or nucleotide-based hybridization methods.

38. The method of claim 37, wherein the detection of the amplicons obtained in step b) is carried out using a microarray.

39. The method of claim 29 for use in detecting contaminations in thrombocyte concentrates.

40. The method of claim 29 for use in detecting infections, in particular systemic infections, selected from the group consisting of sepsis, spontaneous bacterial peritonitis and endocarditis.

41. A diagnostic kit for determining bacteria and fungi contained in a sample material, wherein the kit comprises:
a) a means for enriching bacterial and fungal DNA contained in the sample material from total DNA of the sample material;
b) a means for a multiplex amplification of the enriched bacterial and fungal DNA, wherein the means include at least 20 primer pairs selected from primer pairs of at least two of groups (i) to (vii), wherein:
group (i) comprises at least one primer pair which is suited for the specific amplification of a region of a particular nucleic acid sequence that is specific for a plurality of bacteria families;
group (ii) comprises at least one primer pair which is suited for the specific amplification of a region of a particular nucleic acid sequence that is specific for a plurality of fungus families;
group (iii) comprises at least one primer pair which is suited for the specific amplification of a region of a particular nucleic acid sequence that is specific for a selected bacteria genus;
group (iv) comprises at least one primer pair which is suited for the specific amplification of a region of a particular nucleic acid sequence that is specific for a selected bacteria species;
group (v) comprises at least one primer pair which is suited for the specific amplification of a region of a particular nucleic acid sequence that is specific for the expression of a selected antibiotics or antimycotics resistance;
group (vi) comprises at least one primer pair which is suited for the specific amplification of a region of a particular nucleic acid sequence that is specific for a selected fungus genus; and
group (vii) comprises at least one primer pair for the specific amplification of a region of a particular nucleic acid sequence that is specific for a selected fungus species, and
c) a means for detecting the amplicons obtainable with the primer pairs of b).

42. The kit of claim 41, wherein the at least one primer pair of group (i) is a primer pair which specifically hybridizes to the nucleic acid sequence of the gene for bacterial 16S rDNA.

43. The kit of claim 41, wherein the at least one primer pair of group (ii) is a primer pair which specifically hybridizes to the nucleic acid sequence of the gene for fungal 18S rDNA.

44. The kit of claim 41, wherein the means for amplifying DNA include means to provide the amplicons with a detectable marker during amplification.

45. The kit of claim 41, wherein the means for amplifying DNA include means for performing non-quantitative PCR.

46. The kit of claim 41, wherein the means for amplifying DNA include means for performing real-time quantitative PCR (qPCR).

47. The kit of claim 45, wherein the means for detecting the amplicons obtainable with the primer pairs of b) include agents for producing electrophoresis gels.

48. The kit of claim 45, wherein the means for detecting the amplicons obtainable with the primer pairs of b) include means for performing a microarray.

49. Use of the kit of claim 41 for detecting contaminations in thrombocyte concentrates.

50. Use of the kit of claim 41 for detecting infections, in particular systemic infections, selected from the group consisting of sepsis, spontaneous bacterial peritonitis and endocarditis.
